(19) United States
(12) Patent Application Publication
(10) Pub. No.: US 2010/0029529 A1
(43) Pub. Date: Feb. 4, 2010

(54) Surfactant having chlorine-capturing ability and anti-discoloration ability and chemical formulation containing the same surfactant

(76) Inventors: Ryuji Kikuchi, Saitama (JP); Toshihiro Shinoda, Saitama (JP)

Correspondence Address: WENDEROTH, LIND & PONACK, L.L.P., 1030 15th Street, N.W.,, Suite 400 East, Washington, DC, 20005-1503, US

(21) Appl. No.: 12/588,340

(22) Filed: Oct. 13, 2009

Related U.S. Application Data
(62) Division of application No. 11/640,923, filed on Dec. 19, 2006.

(30) Foreign Application Priority Data
May 9, 2006 (JP) 2006-130301

(51) Int. Cl. A61K 8/41; A61Q 5/02; C11D 1/94

(52) U.S. Cl. 510/123; 510/275

(57) ABSTRACT

An amphoteric surfactant reduces adverse effects of free chlorine and ensures high color retention of dyed hair, dyed keratin fibers and dyed fabrics made of dyed keratin fibers. The surfactant is represented by the following general formula (1):

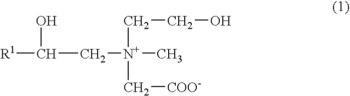

[wherein R¹ is an alkyl or alkenyl group having 10 to 18 carbon atoms].

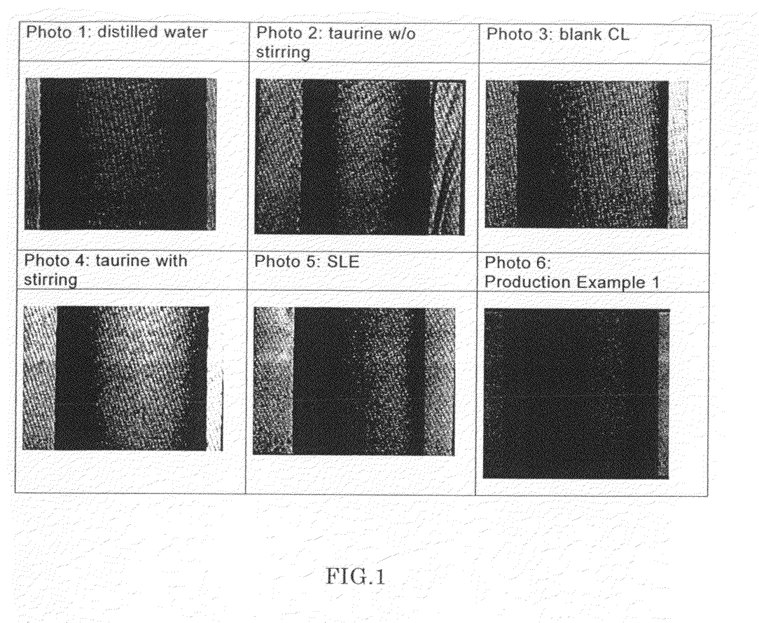

### BACKGROUND OF THE INVENTION

1. Field of the Invention

The present invention relates to a particular type of surfactant that has an ability to capture chlorine and to reduce discoloration of dyed hair or dyed keratin fiber. The present invention also relates to chemical formulations, in particular detergent compositions, that contain the surfactant.

2. Description of the Related Art Chlorine has a bactericidal effect and a bleaching effect and is therefore widely used in chlorine bleaches as well as in disinfectants for use in tap water, swimming pools, and circulatory baths.

The primary objective of chlorinating tap water is to sterilize water to prevent infectious diseases caused by pathogenic microbes present in tap water. The historical fact that prevention of infectious diseases was the driving force behind the widespread use of modern water supply systems has established chlorination as an essential process for water treatment.

Although ultraviolet irradiation and other new sterilization techniques have been developed, chlorination still remains by far the most common technique used to sterilize tap water in many countries other than Brazil, Netherlands, and Switzerland.

In Japan, the water supply law specifies the lower limit for the residual chlorine in tap water at the faucet normally as 0.1 ppm or higher, or as 0.2 ppm or higher when contamination with aqueous pathogenic microbes is suspected. However, the law specifies no upper limit for the residual chlorine. Thus, tap water containing even higher levels of residual chlorine is used for washing purpose during the suitable seasons for the growth of bacteria.

Acceptable free residual chlorine levels in swimming pools and other swimming facilities are specified as 0.4 mg/L (0.4 ppm) or, ideally, 1.0 mg/L (1.0 ppm) or below according to the notification by the director of the department of environmental hygiene, Ministry of Health and Welfare of Japan. The upper limit is defined since high levels of chlorine irritate the skin. Nonetheless, it is practically difficult to maintain a constant level of free residual chlorine throughout the entire swimming pool and there is a significant chance that the swimmers are exposed to a high level of free chlorine.

The residual free chlorine and free chlorine released from combined chlorines, such as chloramines formed by the condensation of free chlorine and a nitrogen compound, may cause damage to the body if they are not removed completely by shower after swimming.

Free chlorine also causes hair damage: Hair often becomes brittle and discolors after swimming in a swimming pool. Hair is more susceptible to free chlorine than is the skin.

We generally shampoo our hair by rubbing the scalp with fingers while pouring 40 to 50° C. warm water. This causes a friction between hair strands and between hair and fingers and, as a result, a physical stress is applied to hair. The study by the present inventors has revealed that water containing only low levels of free chlorine, such as tap water, can cause cuticle peeling and other damage to hair if hair is exposed to such water for an extended period of time. We have also found that hair suffers similar damage when exposed to free chlorine and at the same time to heat or physical stress.

Damage to hair cuticles can expose the internal cortex and the medulla layers of the hair. When polyvalent metals such as calcium penetrate the exposed layers, the hair becomes brittle and easy to break. In addition, it is believed that free chlorine or other oxidants can denature the protein within the hair, making hair dull and causing discoloration.

Japanese Patent Publication No. Hei 06-041409 describes an ophthalmic liquid preparation that uses taurine to mitigate the damage to eye tissue caused by chlorine disinfectants during swimming in swimming pools. Taurine in this preparation acts to capture free chlorine.

Japanese Patent Laid-Open Publication No. 2002-320981 describes a method of improving tap water and improved tap water suitable for drinking, face washing and watering animals and plants. The water is improved by a simple and economical technique: simply adding taurine to tap water. According to the disclosure, taurine “may be used in soap or shampoo, which can be used with tap water to wash face to achieve the same effect.” It is pointed out that cosmetics containing taurine can promote the skin health and have a significant beauty effect on the skin. It is considered that in this technique, taurine reacts with free chlorine to convert it into a chloramine-like product, a combined chlorine, eliminating the problems posed by free chlorine.

In light of these prior art technologies, the present inventors thought of an idea that taurine may be added to a shampoo composition as an agent to capture free chlorine and thought that this would decrease the free chlorine level to which hair was exposed during shampooing, thus preventing damage to hair. Accordingly, the present inventors conducted experiments using different compositions containing varying amounts of chlorine and taurine. The results of the experiments demonstrated that although the addition of large amounts of taurine with respect to chlorine had some effect, the ability of taurine to capture free chlorine was insignificant in warm water or in the presence of physical stresses, or specifically when agitation is applied. Furthermore, taurine was an expensive compound and was not economical.

Thus, there was a need for a chemical formulation that can effectively eliminate free chlorine from shampoo to avoid the hair damage caused by free chlorine.

The increasing popularity of hair dyeing has led to more people enjoying dyeing their hair. As more people use oxidative dyes and bleaches to dye their hair, the need to minimize hair damage and maximize color retention is higher than ever before. While much effort has been devoted to improving hair dye techniques to meet such needs, few techniques have been developed that address the problem of discoloration of dyed hair after shampooing.

Japanese Patent Laid-Open Publication No. 2002-47147 discloses a shampoo composition for use with dyed hair. This composition comprises (A) one or two or more selected from anionic surfactants and amphoteric surfactants and (B) arginine and/or a salt thereof, and (C) has a pH of 4 to 7. The composition however is effective only in a weakly acidic to neutral pH range and its desired effect becomes less than satisfactory near neutral pH. The limitation on the pH often restricts the performance of the anionic surfactant and the amphoteric surfactant used in the composition. A solution to this problem has been sought for.

An amphoteric surfactant represented by the following general formula (1):

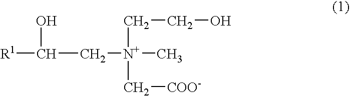

(wherein R¹ is an alkyl or alkenyl group having 10 to 18 carbon atoms) is a compound encompassed by the scope of the invention described in Japanese Patent Laid-Open Publications No. Sho 52-087117, No. Sho 52-085987, and No. Sho 52-126410, and is included in what is represented by the structural formulas presented in these literatures. However, none of these documents mentions the compound of the general formula (1) itself.

Specifically, any of the above patent documents mentions nothing about physical properties of a surfactant composition containing the amphoteric surfactant of the general formula (1), such as the following properties:
1. It has a high compatibility with other surfactants.
2. It can be combined with other surfactants to establish a desired viscosity: The surfactant composition has a unique rheology in that when it is mixed with a certain sulfur-containing anion at a certain ratio, the viscosity of the mixture does not change upon dilution.
3. It has a strong ability to capture chlorine.
4. Despite its relatively high washing power, the surfactant composition causes less discoloration of dyed hair than other surfactants.

### SUMMARY OF THE INVENTION

Accordingly, it is an object of the present invention to provide a surfactant that can reduce the adverse effect of free chlorine and can ensure high color retention of dyed hair, dyed keratin fiber or dyed fabric made of dyed keratin fiber. It is another objective of the present invention to provide a chemical formulation that uses the surfactant.

In an effort to achieve the above-described objects, the present inventors have closely studied a variety of betaine compounds and, as a result, have found that the compound of the general formula (1) in particular exhibits a high surface activity over a broad pH range or in hard water, is highly stable at low temperatures, shows a high compatibility with other surfactants, and can be combined with other surfactants to establish a desired viscosity.

The present inventors have also found that this particular betaine compound can be added to a detergent composition for dyed hair and dyed keratin fiber to not only ensure high color retention of the fiber, but also achieve sufficient washing power, high foaming performance and pleasant refreshing texture.

Not only has the betaine compound been found to be highly compatible with other surfactants, but also it has been proven usable with other surfactants to achieve a wide range of viscosity. In particular, the betaine compound can be mixed with a particular sulfate-based surfactant at a particular ratio to make a liquid detergent that exhibits a unique viscoelastic behavior: The viscosity of the liquid detergent composition does not change when it is diluted two-fold (by weight) with water.

In addition to its unique surface activity, the compound of the general formula (1) has a higher ability to capture free chlorine than taurine, a compound commonly used as a chlorine-capturing agent. The present inventors have found that these properties of the betaine compound of the general formula (1) can be exploited in making chemical formulations, such as post-treatment agents for bleached products, detergent compositions for dyed hair, detergent compositions for dyed hair and anti-chlorine damage shampoos. It is these findings that ultimately led to the present invention.

Accordingly, a first aspect of the present invention concerns an amphoteric surfactant represented by the following general formula (1):

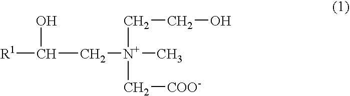

[wherein R¹ is an alkyl or alkenyl group having 10 to 18 carbon atoms].

A second aspect of the present invention concerns a chlorine-capturing agent comprising the amphoteric surfactant of the general formula (1), as well as a cosmetic product and an anti-chlorine damage shampoo that makes use of the chlorine-capturing effect.

A third aspect of the present invention concerns a detergent, a detergent composition for dyed hair, and a detergent composition for dyed keratin fiber that take advantage of the amphoteric surfactant of the general formula (1) that has a relatively high washing power, but nonetheless causes less discoloration of dyed products than other surfactants.

Specifically, the third aspect of the present invention concerns the fact that the amphoteric surfactant of the general formula (1) is highly compatible with amphoteric surfactants other than those represented by the general formula (1), as well as with ampholytic surfactants, semipolar surfactants, anionic surfactants, nonionic surfactants, and cationic surfactants, and the fact that the amphoteric surfactant of the general formula (1) can be combined with surfactants other than those represented by the general formula (1) to improve the viscosity stability (or retention ability) of the solution. The third aspect particularly concerns the fact that when a sulfate-based surfactant or a sulfonic acid-based anionic surfactant is used as the other surfactant component and is mixed with the amphoteric surfactant of the general formula (1) at a specific ratio, the viscosity of the resulting mixture does not decrease when the mixture is diluted with water.

The amphoteric surfactant of the present invention represented by the general formula (1) exhibits a high surface activity over abroad pH range or in hard water, is highly stable at low temperatures, and shows a high compatibility with other surfactants.

The amphoteric surfactant of the general formula (1) has a superb ability to capture free chlorine and can be brought into contact with or mixed with solutions containing dissolved chlorine or materials containing chlorine to capture free chlorine, a strong oxidant, thus eliminating its effect and stabilizing drugs or other chemical agents that are otherwise susceptible to free chlorine. When used with a hydroxycarboxylic acid in a shampoo composition, the amphoteric surfactant of the general formula (1) serves to eliminate free chlorine from water used to wash hair, so that hair is no longer damaged by the chlorine-containing water. Such a shampoo composition can also eliminate chlorine accumulated in hair and effectively reduce damage to hair. As a result, the hair washed by the shampoo suffers less damage from free chlorine in water and retains shine and strength. Therefore, the present invention can provide a superior anti-chlorine damage shampoo.

When used in detergents for dyed hair and dyed keratin fibers, the amphoteric surfactant of the general formula (1) ensures color retention while achieving sufficient washing power and high foaming performance. It also realizes pleasant texture.

The amphoteric surfactant of the general formula (1) can be mixed with other surfactants to achieve a wide range of viscosity. In particular, the amphoteric surfactant can be mixed with a particular sulfate-based surfactant at a particular ratio to make a liquid detergent that exhibits a unique viscoelastic behavior: The viscosity of the liquid detergent composition does not change when it is diluted two-fold (by weight) with water. Such a liquid detergent is easy to apply to surfaces that require washing.

### BRIEF DESCRIPTION OF THE DRAWING

FIGURE includes SEM photographs of cuticles.

### DETAILED DESCRIPTION OF PREFERRED EMBODIMENTS

The amphoteric surfactant of the general formula (1) can be produced by the following processes: Reacting N-hydroxyethyl glycine with an epoxy alkane and subsequently applying a methylation agent to make a desired betaine; reacting sarcosine with an epoxy alkane and subsequently applying bromohydrin to make a desired betaine; or reacting N-methylethanolamine with an epoxy alkane and subsequently applying monochloroacetic acid to make a desired betaine, as shown below:

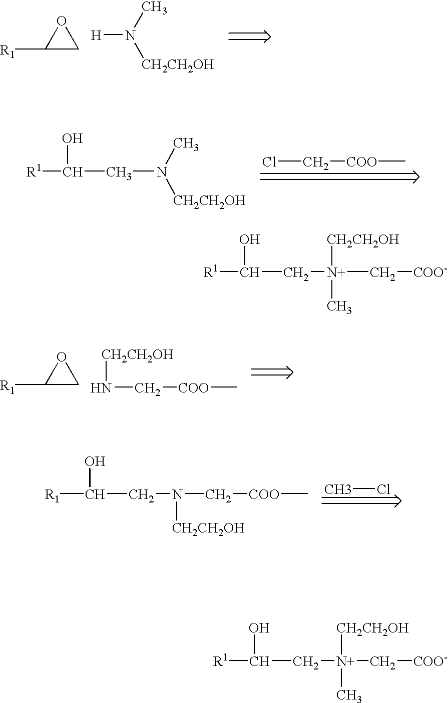

[wherein in the formula (1), R¹ is an alkyl or alkenyl group having 10 to 18 carbon atoms].

The betainization often leads to the generation of inorganic salt by-products (generally sodium chloride) or results in an amine of the following general formula (2) and an aminocarboxylic acid of the general formula (3) remaining in the reaction product:

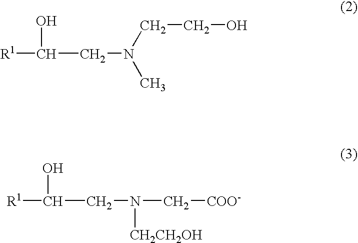

[wherein in the formulas (2) and (3), R¹ is as defined above].

The inorganic salts may cause a decreased solubility of cationized polymers or other problems and are thus preferably removed by known techniques, such as electrodialysis or RO membrane.

The compound of the general formula (2) or (3) is poorly soluble in water and may affect the low temperature stability when present in abundance. For this reason, the compound of the general formula (1) and the compound of the general formula (2) or (3) are preferably present at a ratio (by weight) of 99:1 to 80:20.

Preferred examples of the amphoteric surfactant of the general formula (1) include
N-(2-hydroxydodecyl)-N-hydroxyethyl-N-methylglycine,
N-(2-hydroxydecyl)-N-hydroxyethyl-N-methylglycine, and
N-(2-hydroxyundecyl)-N-hydroxyethyl-N-methylglycine.

Similarly to amphoteric surfactants and ampholytic surfactants other than those represented by the general formula (1), the amphoteric surfactant of the general formula (1) becomes thicker when used with an anionic surfactant.

When the amphoteric surfactant of the present invention is used in combination with an anionic surfactant, it shows different viscosity behaviors from other amphoteric/ampholytic surfactants in the following two aspects:
(A) The amphoteric surfactant of the present invention leads to a higher viscosity than do other amphoteric/ampholytic surfactants when added to the anion in relatively small amounts.
(B) When the amphoteric surfactant of the present invention is mixed with a sulfate-based and/or a sulfonic acid-based anionic surfactant at a particular ratio to make a liquid detergent of a desired viscosity, the viscosity does not change upon dilution.

The property described in (A) makes the amphoteric surfactant of the general formula (1) suitable for use as a thickener since the amphoteric surfactant can effectively increase the viscosity of the mixture when added to a liquid detergent composition containing mainly an anionic surfactant in smaller amounts as compared to other amphoteric/ampholytic surfactants.

The property described in (B) is achieved when the betaine compound of the present invention and the sulfate-based or sulfonic acid-based anion are mixed at a ratio (by weight) of 10:10 to 14:6. The viscosity established by mixing the components in this manner does not change when the composition is diluted two-fold (by weight) with water.

When the ratio of the two components falls outside this range, the viscosity of the composition changes upon dilution.

Examples of the sulfate-based or sulfonic acid-based anion used for this purpose include polyoxyethylene alkyl ether sulfates, triethanolamine lauryl sulfates, alkyl benzene sulfonates, and alkane sulfonates.

We now describe the ability of the amphoteric surfactant of the general formula (1) of the present invention to capture chlorine.

Chlorine in water can be measured by techniques such as diethyl-p-phenylenediamine (DPD) method: Amounts of free chlorine, combined chlorine, and total chlorine as the sum of the free chlorine and the combined chlorine can be determined.

The term “free chlorine” as used herein is defined as the concentration of residual chlorine present in water as dissolved chlorine gas (Cl₂), hypochlorous acid (HOCl) and hypochlorous acid ion (OCl⁻). Free chlorine is the most powerful oxidant and can seriously damage hair.

The term “combined chlorine” as used herein refers to residual chlorine in water that is chemically bound to ammonia or organic amines present in natural or contaminated water. As previously described, taurine is considered to capture dissolved chlorine as combined chlorine by reacting with free chlorine to form chloramine-like compound. While combined chlorine has less disinfectant activity than free chlorine, it releases free chlorine in response to changes in the surrounding chemical environment. The amount of total chlorine is the sum of the amounts of the free chlorine and the combined chlorine.

A typical DPD method is performed in the following manner: DPD is first added to water to measure free chlorine. Potassium iodide is then added to cause combined chlorine to be released as free chlorine and the total chlorine is measured by DPD. The amounts of the total chlorine and the free chlorine were used to determine the combined chlorine.

The present inventors examined various compounds for the ability to capture dissolved chlorine and found that the amphoteric surfactants of the general formula (1) were particularly effective in capturing and decreasing free chlorine. The measurement of the total chlorine indicated that not all of the chlorine used was collected.

The reason for this is believed to be that the amphoteric surfactant of the general formula (1) more strongly binds to free chlorine than common chlorine-capturing agents such as taurine. This combined chlorine is less likely to be released as free chlorine in response to changes in the surrounding chemical environment, such as addition of potassium iodide solution, which is added to determine total chlorine by causing combined chlorine to be released as free chlorine.

This suggests that the amphoteric surfactant of the general formula (1) is a powerful agent to remove free chlorine.

Chlorine-capturing agents comprising the amphoteric surfactant of the present invention represented by the general formula (1) can be used to make detergent compositions, anti-chlorine damage shampoos, cosmetics and post-treatment agents for bleached products (These may be collectively referred to as “chemical formulations,” hereinafter).

We now describe detergent compositions, one of the chemical formulations containing the chlorine-capturing agent of the present invention.

The detergent composition preferably contains 1.0 wt % or more of the chlorine-capturing agent of the present invention, although the amount may vary depending on how much free chlorine is present in products that are to be washed by the detergent.

The detergent may be provided in any of the following forms: liquid, cream, foam and solid.

While the chlorine-capturing agent of the present invention, which itself acts as an amphoteric surfactant, can be used alone to make a detergent composition, it may be combined with anionic surfactants, nonionic surfactants, semipolar surfactants, amphoteric surfactants other than those represented by the general formula (1), ampholytic surfactants or cationic surfactants to modify the washing performance, foaming performance, and using sensation of the composition.

In addition, ingredients used in common cosmetics may be added in amounts that do not affect the advantages of the present invention.

We now describe in detail how the chlorine-capturing agent of the present invention can be used in anti-chlorine damage shampoos.

As previously described, water containing low levels of chlorine, such as tap water, causes the cuticle to peel away from hair when the hair is exposed to such water for a prolonged period of time. Under this condition, the peeling of cuticle can be substantially prevented by adding a conventional chlorine-capturing agent, such as taurine, to tap water. The ability of taurine to capture chlorine is high enough to prevent peeling of cuticle in water containing higher levels of chlorine than tap water, but only for a short period of time. However, taurine can no longer prevent peeling of cuticle when the hair is immersed in warm tap water at 40 to 50° C. or when hair is immersed in a test solution agitated to simulate the physical stress that applies during shampooing.

Although shampoo compositions composed solely of taurine or other conventional agents for capturing dissolved chlorine cannot prevent hair damage under physical stresses, such as shampooing in a warm water, shampoo compositions using the surfactant of the general formula (1) can prevent hair damage under heat or stresses by effectively removing chlorine from water. This is believed to be because the chlorine-capturing agent comprising the surfactant of the general formula (1) has a more powerful ability to remove chlorine than taurine and other conventional chlorine-capturing agents.

The anti-chlorine damage shampoo preferably contains the chlorine-capturing agent comprising the surfactant of the general formula (1) in an amount of 1.0 wt % or more, and more preferably in an amount of 5.0 wt % or more, while the amount may vary depending on how much dissolved chlorine is present in water used for shampooing. Though the chlorine-capturing agent may be added in any suitable amount, the agent added in amounts exceeding 30 wt % makes it difficult to obtain a stable liquid composition and is not preferred.

We now describe the ability of the amphoteric surfactant of the general formula (1) to prevent discoloration of dyed hair, dyed pubic hair, dyed keratin fibers, and fabrics made of dyed keratin fiber (These are referred to as “dyed keratin products,” hereinafter).

The detergent composition containing the amphoteric surfactant of the general formula (1) can be used to wash dyed keratin products with little discoloration. The detergent composition for dyed keratin products preferably contains the betaine compound of the general formula (1) in an amount of 0.1 to 50 wt %. The betaine compound cannot effectively prevent discoloration of the dyed keratin products when present in an amount of less than 0.1%, while it results in unpleasant using sensation and a decreased stability of the preparation when present in an amount of 50 wt % or more. Too much or too little of the betaine compound causes unpleasant using sensation especially in hair shampoos for dyed hair.

Though the underlying mechanism is still unclear, it is believed that the amphoteric surfactant of the general formula (1) serves to tighten tissue. For example, it is assumed that much of the dye is attached to hair by clinging to the scaffold provided by the cuticle gaps. The detergent of the present invention, having a stronger ability to hold cuticles together as compared to other surfactants, can prevent the dye from readily coming off hair during shampooing.

When the amphoteric surfactant of the general formula (1) is used in combination with commonly used anionic surfactants, nonionic surfactants, amphoteric surfactants, ampholytic surfactants or semipolar surfactants, it is preferably used in an amount of 50 wt % or more with respect to the total surfactants to optimize the ability to prevent discoloration of dyed keratin products.

We now describe in detail a secondary component that can be used in the post-treatment agents for bleached products, the detergent compositions for dyed hair, the detergent compositions for died keratin fibers, the anti-chlorine damage shampoos and other chemical formulations containing the amphoteric surfactant of the general formula (1).

If necessary, the chemical formulation of the present invention may contain at least one of the following ingredients: extracts of animals, plants, fish and shellfish or microorganisms, powder ingredients, liquid oils and fats, solid oils and fats, waxes, hydrocarbons, higher fatty acids, higher alcohols, esters, silicone, anionic surfactants, cationic surfactants, amphoteric surfactants, nonionic surfactants, humectants, water-soluble polymers, thickeners, coating agents, UV absorbents, antiphlogistics, metal-chelating agents, lower alcohols, sugars, amino acids, organic amines, synthetic resin emulsions, pH adjusters, skin nutrients, vitamins, antioxidants, antioxidant aids, perfumes and deep sea water.

Examples of the extracts of animals, plants, fish and shellfish or microorganisms for use in the chemical formulation of the present invention include extracts of tea leaves, aloe plants, ginkgo leaves, Japanese green gentian, mugwart, garlic, Scutellariae Radix, rosemary, luffa, placenta, lactobacillus culture and seaweeds.

Examples of the powder ingredients for use in the chemical formulation of the present invention include inorganic powders, such as talc, kaolin, mica, sericite, Muscovite, Phlogopite, Synthetic mica, Lepidolite, Biotite, Lithia mica, vermiculite, magnesium carbonate, zirconium silicate, aluminum silicate, barium silicate, calcium silicate, zinc silicate, magnesium silicate, strontium silicate, tungstic acid metal salts, magnesium, silica, zeolite, barium sulfate, baked calcium sulfate (calcined gypsum), calcium phosphate, fluorine apatite, hydroxyapatite, ceramic powders, active carbon, medicinal carbon, metal soaps (e.g., zinc myristate, calcium palmitate and aluminum stearate) and boron nitride; and organic powders, such as polyamide resin powder (i.e., nylon powder), polyethylene powder, polymethylmethacrylate powder, polystyrene powder, resin powders of copolymers of styrene and acrylic acid, benzoguanamine resin powder, polyethylene tetrafluoride powder and cellulose powder.

Examples of the liquid fats and oils for use in the chemical formulation of the present invention include avocado oil, camellia oil, grapeseed oil, turtle oil, macadamia ternifolia seed oil, corn germ oil, mink oil, olive oil, sunflower oil, rapeseed oil, egg yolk oil, sesame oil, apricot kernel oil, wheat germ oil, camellia kissi oil, castor seed oil, linseed oil, safflower seed oil, cotton seed oil, perilla oil, soybean oil, peanut oil, camellia sinensis oil, Japanese torreya oil, rice bran oil, Chinese paulownia oil, Japanese paulownia oil, jojoba seed oil, germ oil, triglycerol, trioctanoic acid glyceride and triisopalmitic acid glyceride.

Examples of the solid fats and oils for use in the chemical formulation of the present invention include cacao seed butter, coconut oil, horse fat, hydrogenated coconut oil, palm oil, beef tallow, mutton tallow, hydrogenated tallow, palm kernel oil, lard, beef bone tallow, rhus succedanea fruit wax, hydrogenated oil, beef leg tallow, rhus succedanea fruit and hydrogenated castor seed oil. Example of the waxes for use in the chemical formulation of the present invention include beeswax, candelilla wax, cotton wax, carnauba wax, bayberry wax, purified insect wax, whale wax, montan wax, bran wax, lanolin, kapok wax, lanolin acetate, liquid lanolin, sugarcane wax, lanolin fatty acid isopropyl ester, hexyl laurate, reduced lanolin, jojoba wax, hard lanolin, shellac wax, POE-lanolin alcohol ether, POE-lanolin alcohol acetate, POE-cholesterol ether, lanolin fatty acid polyethylene glycol and POE-hydrogenated lanolin alcohol ether.

Examples of the hydrocarbon oils for use in the formulation of the present invention include liquid paraffin, ozocerite, squalene, pristane, paraffin, ceresin, squalane, Vaseline and microcrystalline wax. Examples of the higher fatty acids include lauric acid, myristic acid, palmitic acid, stearic acid, behenic acid, oleic acid, 12-hydroxystearic acid, undecylenic acid, tall oil fatty acid, coconut oil fatty acid, palm fatty acid, palm kernel fatty acid, isostearic acid, linoleic acid, linolenic acid, eicosapentaenoic acid and docosahexaenoic acid.

Examples of the synthetic ester oils for use in the chemical formulation of the present invention include isopropyl myristate, cetyl octanoate, octyldodecylmyristate, isopropyl palmitate, butyl stearate, hexyl laurate, myristyl myristate, decyl oleate, hexyldecyl dimethyloctanoate, cetyl lactate, myristyl lactate, lanolin acetate, isocetyl stearate, isocetyl isostearate, cholesteryl 12-hydroxystearate, ethylene glycol di-2-ethylhexanoate, dipentaerythritol fatty acid esters, neopentyl glycol caprate, diisostearyl malate, glyceryl di-2-heptylundecanoate, trimethylolpropane tri-2-ethylhexanoate, trimethylolpropane triisostearate, pentaerythritol tetra-2-ethylhexanoate, glyceryl tri-2-ethylhexanoate, trimethylolpropane triisostearate, cetyl 2-ethylhexanoate, 2-ethylhexyl palmitate, glyceryl trimyristate, tri-2-heptylundecanoate glyceride, castor oil fatty acid methyl esters, oleic acid oil, cetostearyl alcohol, acetoglyceride, 2-heptylundecyl palmitate, diisobutyl adipate, 2-octyldodecyl N-lauroyl-L-glutamate, 2-heptylundecyl adipate, ethyl laurate, 2-ethylhexyl sebacate, 2-hexyldecyl myristate, 2-hexyldecyl palmitate, 2-hexyldecyl adipate, diisopropyl sebacate, 2-ethylhexyl succinate, ethyl acetate, butyl acetate, amyl acetate and triethyl citrate.

Examples of the silicones for use in the chemical formulation of the present invention include dimethyl silicone oil, methylpolysiloxane, octamethyltrisiloxane, highly polymerized methylpolysiloxane, decamethylpolysiloxane, dodecamethylpolysiloxane tetramethyltetrahydrogenpolysiloxane, dimethylsiloxane/methyl(polyoxyethylene)siloxane copolymer, dimethylsiloxane/methyl(polyoxyethylene)siloxane/methyl(polyoxypropylene)siloxane copolymer and amino-modified silicone.

Examples of the anionic surfactants for use in the chemical formulation of the present invention include fatty acid soaps, such as soap base, sodium laurate, sodium palmitate and potassium soap of coconut fatty acid; higher alkyl sulfates, such as sodium lauryl sulfate, potassium lauryl sulfate and triethanolamine lauryl sulfate; alkyl ether sulfates, such as triethanolamine POE-lauryl sulfate and sodium POE-lauryl sulfate; N-acylamino acid salts, such as sodium lauroyl sarcosinate, sodium lauroyl-β-alanine, sodium lauroyl-N-methyl-β-alanine, monosodium N-lauroyl glutamate, disodium N-stearoyl glutamate, monosodium N-myristoyl-L-glutamate, diethanolamine N-palmitoyl aspartate, potassium soap of coconut fatty acid and silk amino acid and sodium lauroyl alanine; amidosulfonic acid salts of higher fatty acids, such as sodium N-myristoyl-N-methyl taurate, sodium cocoyl methyl taurate and sodium lauroyl methyl taurate; phosphates, such as sodium POE-oleyl ether phosphate, POE-stearyl ether phosphoric acid and sodium POE-lauryl amidoether phosphate; sulfosuccinates, such as sodium di-2-ethylhexylsulfosuccinate, sodium monolauroyl monoethanol amide polyoxyethylene sulfosuccinate and sodium lauryl polypropylene glycol sulfosuccinate; alkylbenzene sulfonates, such as sodium linear dodecylbenzenesulfonate, triethanolamine linear dodecylbenzenesulfonate and linear dodecylbenzenesulfonic acid; higher fatty acid sulfates, such as hydrogenated palm oil fatty acid glycerin sodium sulfate; sulfated oils, such as turkey red oil; α-olefin sulfonates; higher fatty acid ester sulfonates; secondary alcohol sulfates; higher fatty acid alkylol amide sulfates; sodium lauroyl monoethanolamide succinate; and sodium caseinate.

Examples of the cationic surfactants for use in the chemical formulation of the present invention include alkyltrimethyl ammonium salts, such as stearyl trimethyl ammonium chloride, lauryl trimethyl ammonium chloride and lauryl trimethyl ammonium bromide; alkylpyridinium salts, such as dialkyldimethyl ammonium salts (e.g., distearyl dimethyl ammonium chloride); and alkyl dimethylbenzyl ammonium salts, such as cetylpyridinium chloride, benzethonium chloride and benzalkonium chloride.

Examples of the ampholytic surfactants for use in the chemical formulation of the present invention include alkylsulfo betaine-type ampholytic surfactants and amidosulfo betaine-type ampholytic surfactants.

Of the ampholytic surfactants, amidosulfo betaine-type ampholytic surfactants represented by the following general formula (4) are particularly suitable for use in the detergent composition for hair color, one of the functional chemical formulations of the present invention, since these surfactants can only weakly discolor hair in a low pH range:

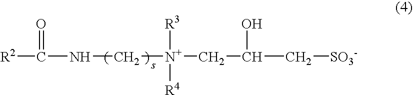

[wherein R² is an alkyl or alkenyl group having 10 to 18 carbon atoms; R³ and R⁴ are each a substituent selected from a hydrogen atom, a methyl group and an ethyl group; and s is an integer of 2 or 3].

Examples of the amidosulfobetaines include lauroylamidopropyl hydroxysulfobetaine and cocoamidopropyl hydroxysulfobetaine.

Examples of the amphoteric surfactants for use in the chemical formulation of the present invention include amidoamine-based amphoteric surfactants, such as 2-undecyl-N-carboxymethyl-N-hydroxyethylimidazolinium betaine, N-lauroyl-N′-carboxymethyl-N′-hydroxyethylethylenediamine sodium salt and N-cocoacyl-N′-carboxyethyl-N′-hydroxyethylethylenediamine sodium salt; amidoacetic acid betaine-type amphoteric surfactants, such as coconut fatty acid amidopropyl betaine and myristic acid amidopropyl betaine; alkylamino acid-type amphoteric surfactants, such as N-lauryl-β-alanine, POE-N-lauryl-β-alanine and N-lauryl-iminodiacetic acid; and alkylacetic acid betaine-type amphoteric surfactants.

Examples of the semipolar surfactants for use in the chemical formulation of the present invention include amineoxide-type semipolar surfactants, such as lauryl trimethylamineoxide and lauroylamidopropylamine oxide.

Examples of the nonionic surfactants for use in the chemical formulation of the present invention include glycerol fatty acid esters, such as glyceryl monostearate, self-emulsifying glyceryl monostearate and glyceryl monoisostearate; polyoxyethylene-glycerol fatty acid esters, such as monostearic acid and POE-glyceryl monooleate POE-glyceryl; polyglycerol fatty acid esters, such as diglyceryl monostearate, tetraglyceryl tristearate and decaglyceryl pentastearate; sorbitan fatty acid esters, such as sorbitan monolaurate, sorbitan sesquistearate and sorbitan monooleate; polyoxyethylene-sorbitan fatty acid esters, such as POE-sorbitanmono-coconut oil fatty acid ester, POE-sorbitan tristearate and POE-sorbitan trioleate; polyoxyethylene-sorbitol fatty acid esters, such as POE-sorbitol monolaurate and POE-sorbitol tetraoleate; polyethylene glycol fatty acid esters, such as polyethylene glycol monolaurate, polyethylene glycol monostearate, polyethylene glycol monooleate and polyethylene glycol distearate; polyoxyethylene-alkyl ethers, such as POE-lauryl ether, POE-cetyl ether and POE-stearyl ether; polyoxyethylene polyoxypropylene-alkyl ethers, such as POE-POP cetyl ether and POE-POP decyltetradecyl ether; polyoxyethylene-alkylphenyl ethers, such as POE-nonylphenyl ether, POE-octylphenyl ether and POE-branched octylphenyl ether; polyoxyethylene-alkyl amines, such as POE-stearyl amine and POE-oleyl amine; fatty acid alkanolamides, such as coconut oil fatty acid diethanolamide, coconut oil fatty acid monoethanolamide, lauric acid diethanolamide and palm kernel oil fatty acid diethanolamide; polyoxyethylene-alkanolamides, such as POE-lauric acid monoethanolamide, POE-coconut fatty acid monoethanolamide, POE-tallow fatty acid monoethanolamide, POP-lauric acid monoisopropanolamide, POE-POP-branched fatty acid monoethanolamide, compounds represented by the following general formula (5) or (6):

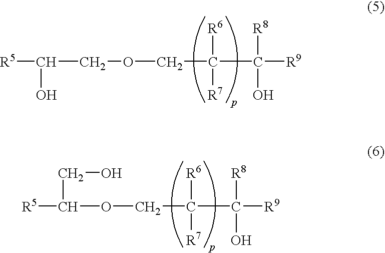

[wherein R⁵ is an alkyl group having 6 to 20 carbon atoms; R⁶, R⁷, R⁸ and R⁹ are each independently a hydrogen atom or an alkyl group having 1 to 3 carbon atoms; and p is an integer of 0 to 3]; acetylene glycol; POE-acetylene glycol; POE-lanoline; POE-lanoline alcohols; POE-castor seed oil; POE-hydrogenated castor seed oil; POE-phytosterols, POE-cholestanols; and POE-nonylphenylformaldehyde condensates.

Examples of the humectants for use in the chemical formulation of the present invention include glycerol, propylene glycol, 1,3-butylene glycol, sorbitol, sodium lactate, pyrrolidonecarboxylic acids and salts thereof. Examples of the water-soluble polymers include guar gum, quince seed, pectin, gelatin, xanthan gum, methylcellulose, ethylcellulose, hydroxyethylcellulose, hydroxypropylcellulose, carboxymethylcellulose and salts thereof, alginates, polyvinyl alcohols, carboxyvinyl polymers, sodium polyacrylates, bentonite, chitin/chitosan derivatives, hyaluronic acid and salts thereof and collagen and derivatives thereof.

Examples of the thickeners for use in the chemical formulation of the present invention include coconut oil fatty acid monoethanolamide, lauric acid diethanolamide, lauric acid isopropanolamide, polyoxyethylene-coconut oil fatty acid monoethanolamide and polyoxypropylene-coconut oil fatty acid monoethanolamide. Examples of the coating agents for use in the cosmetic of the present invention include polyvinyl alcohols, polyvinyl pyrrolidone, cationic cellulose and silicone.

Examples of the UV absorbents for use in the chemical formulation of the present invention include benzophenone derivatives, such as 2-hydroxy-4-methoxybenzophenone, 2-hydroxy-4-methoxybenzophenone-5-sulfonic acid and salts thereof and dihydroxydimethoxybenzophenone, para-aminobenzoic acid, para-aminobenzoic acid derivatives, such as ethyl para-aminobenzoate, ethyl para-methoxycinnamate, isopropyl para-methoxycinnamate, octyl para-methoxycinnamate, methoxycinnamic acid derivatives, salicylic acid derivatives, such as octyl salicylate and phenyl salicylate, urocanic acid and derivatives thereof, 4-tert-butyl-4′-methoxydibenzoylmethane, 2-(hydroxyl-5′-methylphenyl)benzotriazol, and methyl anthranilate.

Examples of the antiphlogistics for use in the chemical formulation of the present invention include glycyrrhizic acid and derivatives thereof, glycyrrhetinic acid and derivatives, allantoin, hydrocortisone acetate and azulene. Examples of the metal-chelating agents for use in the chemical formulation of the present invention include ethylenediamine tetraacetate and sodium salts thereof, phosphoricacid, citric acid, ascorbicacid, succinic acid, gluconic acid, sodium polyphosphate and sodium metaphosphate.

Examples of lower alcohols for use in the chemical formulation of the present invention include ethanol, propylalchol, ethylene glycol and diethylene glycol. Examples of the sugars include glucose, lactose, sucrose, starches, carboxymethyl starch and cyclodextrin.

Examples of amino acids for use in the chemical formulation of the present invention include aspartic acid and salts thereof, alanine, arginine, lysine and salts thereof, glycine, cystine, threonine, serine, methionine and taurine. Examples of organic amines include monoethanolamine, diethanolamine, triethanolamine, diisopropanolamine and triethylamine.

Examples of the synthetic resin emulsions for use in the chemical formulation of the present invention include polyacrylic acid ester copolymers and polyvinyl acetate. Examples of the pH adjusters include citric acid, hydrochloric acid, sulfuric acid, phosphoric acid, sodium hydroxide and ammonia. Examples of the skin nutrients include vitamins A, B1, B2, B6, E and derivatives thereof, pantothenic acid and derivatives thereof and biotin.

Examples of the antioxidants for use in the chemical formulation of the present invention include vitamin E, dibutylhydroxytoluene, butylhydroxyanisole and gallates. Examples of the antioxidant aids include ascorbic acid, phytic acid, cephalin and maleic acid. Ingredients other than those described above may also be added.

### Examples

Different amphoteric surfactants of the general formula (1) were prepared and were evaluated for their performance.

### Production Example 1

### N-(2-hydroxyalkyl)-N-hydroxyethyl-N-methylglycine

504.0 g of N-Me-monoethanolamine (1.05 equivalents of epoxy resin) were placed in a 2L four-necked flask and were heated to 80° C. To this flask, 1256.8 g (6.39 mol) of a C12, 14 epoxy resin (AOEX24, DG-009, DAICEL) were added dropwise over 3 hours and the mixture was stirred overnight at 80° C. Subsequently, the reaction mixture was analyzed by gas chromatography to confirm that no residual epoxy resin was detected. While the mixture was maintained at 80° C., excessive amine was removed by a vacuum pump. This resulted in 1735 g of N-2-hydroxyalkyl-N-2-hydroxyethyl-N-methylamine.

To a 5L four-necked flask, 810.1 g (2.98 mol) of N-2-hydroxyalkyl-N-2-hydroxyethyl-N-methylamine and 1961 g water were added and the mixture was heated to 70° C.. Subsequently, an 80% aqueous solution of monochloroacetate (493.0 g, 1.4 equivalents of the amine) and 400 g of 48% NaOH (1.2 equivalents of the monochloro acetate) were added dropwise over 2 hours in a pH range of 7 to 8. The resulting mixture was heated to 97° C. and was aged for 17 hours while 40 g of 48% NaOH (0.1 equivalents of the monochloro acetate) was added as required in a pH range of 7 to 8. Subsequently, the mixture was cooled to room temperature and water was added to make 3775 g of an aqueous solution of N-2-hydroxyalkyl-N-2-hydroxyethyl-N-methylglycine. Dried residue=35.4%, NaCl=6.3%, Active ingredient=(Dried residue —NaCl)=29.1%, Glycolic acid=2.8%, Yield of betaine as determined by HPLC (UV)=92%.

H¹-NMR of the product of Production Example 1 obtained in heavy water:

| | |
|---|---|
| 0.9 ppm | 3H |
| 1.3-1.5 ppm | 20H |
| 3.4 ppm | 3H |
| 3.5-4.3 ppm | 9H |

### Examples 1 and 2, and Comparative Examples 1 Through 4

### Viscosity Enhancement

Different two-component liquid detergent compositions were formulated by mixing the respective components in the proportions shown in Tables 1 and 2 below. The viscosity of each composition was measured at 25° C. by a type-B viscometer. The proportions are by weight and of pure components.

TABLE 1

| | | Comparative | |
|---|---|---|---|
| | Example | Example | |
| | 1 | 1 | 2 |
| SLES | 16 | 16 | 16 |
| Amphoteric surfactant of Production | 4 | | |
| Example 1 | | | |
| Lauroylamidopropyl acetic acid | | 4 | |
| betaine | | | |
| Lauryl acetic acid betaine | | | 4 |
| pH conditioner + purified water | pH = 7.0 | pH = 7.0 | pH = 7.0 |
| | to 100 | to 100 | to 100 |
| Viscosity (mPa · s) | 4333 | 1550 | 828 |

20% SLES (POE(3) sodium lauryl ether sulfate, Emal 20C, KAO Co. Ltd.) has a viscosity of 11 mPa·s.

A comparison between Examples and Comparative Examples indicates that the amphoteric surfactant of the present invention can achieve a higher viscosity in smaller amounts as compared to the other amphoteric surfactants.

TABLE 2

| | | Comparative | |
|---|---|---|---|
| | Example | Example | |
| | 2 | 3 | 4 |
| Potassium soap of coconut fatty acid | 10 | 10 | 10 |
| Amphoteric surfactant of Production | 10 | | |
| Example 1 | | | |
| Lauroylamidopropyl acetic acid | | 10 | |
| betaine | | | |
| Lauryl acetic acid betaine | | | 10 |
| pH conditioner + purified water | pH = 10 | pH = 10 | pH = 10 |
| | to 100 | to 100 | to 100 |
| Viscosity (mPa · s ) | 15400 | 15 | 28.7 |

Although it is generally considered difficult to increase the viscosity of surfactants having carboxylic acid hydrophilic groups, the results of Table 2 indicate that the amphoteric surfactant of the present invention can significantly increase the viscosity of fatty acid salts, which are carboxylic acid anions.

### Example 3

### Viscosity Retention After Dilution

The amphoteric surfactant of Production Example 1 was mixed with SLES (POE(3) sodium lauryl ether sulfate, Emal 20C, KAO Co. Ltd.) at a ratio of 12:8 to make a mixture with a total surfactant concentration of 20wt %. Theinitial viscosity (mPa·s) of the mixture was measured and the mixture was diluted with distilled water to the concentrations shown in Table 3. The viscosity (mPa·s) at each concentration was determined.

TABLE 3
The ratio in the table = amphoteric surfactant of Production
Example 1: SLES (by weight)

| | Concentration | | | | | | | |
|---|---|---|---|---|---|---|---|---|
| | 20% | 18% | 16% | 14% | 12% | 10% | 8% | 6% |
| Viscosity 12:8 | 939 | 899 | 1015 | 1192 | 1537 | 1984 | 1774 | 368.8 |
| Viscosity 4:16 | 4046 | 795 | 96 | | | | | |

As shown in Table 3, the viscosity of the 12:8 mixture showed a slight increase as the mixture was increasingly diluted until two-fold (10% conc.). This phenomenon is observed when the ratio of the amphoteric surfactant of the present invention to a sulfate-based anion, such as POE-lauryl ether sulfate, or a sulfonic acid-based anion remains within the range of 10:10 to 14:6. When the ratio falls outside this range, such viscoelastic behavior is lost and the viscosity of the mixture decreases at any dilution.

### Example 4

### Ross-Miles Foaming Test

Using the Ross-Miles method, the amphoteric surfactant of Production Example 1 and lauroylamidopropyl betaine (Softazoline LPB, Kawaken Fine Chemicals Co. Ltd.) were analyzed for their foaming performance at various pHs (surfactant conc.=0.25%, 40° C.)

TABLE 4

| | | PH | | | | | | |
|---|---|---|---|---|---|---|---|---|
| | | 4 | 5 | 6 | 7 | 8 | 9 | 10 |
| Foam | Betaine compound of | 196 | 190 | 188 | 193 | 190 | 188 | 189 |
| height | Production Example 1 | | | | | | | |
| (mm) | Lauroylamidopropyl acetic | 175 | 177 | 180 | 182 | 181 | 179 | 177 |
| | acid betaine | | | | | | | |

As can be seen from Table 4, the amphoteric surfactant of Production Example 1 showed a significant foaming performance over the entire pH range tested (pH=4-10). The results demonstrate a higher foaming performance of the amphoteric surfactant of Production Example 1 as compared to Softazoline LPB, an amidopropyl betaine.

### Example 5, and Comparative Examples 5 and 6

### Prevention of Discoloration

Samples were prepared according to the table below and were evaluated for the following properties concerning the betaine of the present invention: low temperature stability, hair color discoloration and hair manicure discoloration. The total surfactant amount in each sample was 15 wt %.

### Storage Stability at −5° C. for One Day

Storage stability at the temperature was evaluated on the following scale: A circle indicates that the sample remained a clear uniform liquid; a triangle indicates that the sample became opaque; and a cross indicates that the sample solidified.

Hair Color Discoloration

A strand of white hair (BM-W, Beaulux Co. Ltd., length=10 cm, weight=1 g) was thoroughly washed with a 2% aqueous solution of POE(3) sodium lauryl ether sulfate (Emal 20C, KAO Co. Ltd.) and was dried with a hair drier.

The dried hair strand was dyed with a commercial hair dye (MA CHERIE Lasting Hair Color, Sheer ash color, SHISEIDO Co. Ltd.) according to the manufacturer's instruction. The hair was then thoroughly washed with water and was dried with a hair drier.

The dyed and dried hair strand was placed in a 100 ml bottle. Meanwhile, a test composition was diluted with distilled water to make a 0.5% aqueous solution. 100 ml of this solution was poured into the bottle. The bottle was then sealed with a cap and agitated for 2 days at room temperature.

Following the agitation period, the bottle was uncapped and the dyed hair strand was pulled out. The hair strand was then washed thoroughly with water and was dried with a hair drier. The dried hair strand was analyzed by a differential calorimeter (Spectrophotometer SE 2000, NIPPON DENSHOKU Co. Ltd.) to determine the color difference (ΔE). A smaller ΔE indicates a higher ability to prevent discoloration of hair.

### Hair Manicure Discoloration

Hair strands, including gray hair strands, obtained from Chinese subjects were bundled together with the cuticles aligned in one direction to make an approximately 30 cm-long hair bundle weighing approximately 20 g. The hair bundle was thoroughly washed with a 2% aqueous solution of POE(3) sodium lauryl ether sulfate (Emal 20C, KAO Co. Ltd.) and was dried with a hair drier.

The dried hair bundle was dyed with a commercial hair dye (SALON de PRO Hair Manicure Speedy, honey brown color, DARIYA Co. Ltd.) according to the manufacturer's instruction. The hair was then thoroughly washed with water and was dried with a hair drier.

100 mg hair (±1 mg or less error, about 60 hairs) was separated from the dyed and dried hair bundle and was placed in a 20 ml bottle. Meanwhile, a test composition was diluted with distilled water to make a 0.5% aqueous solution. 20 ml of this solution was poured into the bottle. The bottle was then sealed with a cap and was left for 14 hours at room temperature.

After 14 hours, the bottle was uncapped and 15 ml of the solution was collected from the sample bottle using a pipette and were analyzed by a differential calorimeter (Model 1001DP, NIPPON DENSHOKU Co. Ltd.) to determine the color difference (ΔE). A smaller ΔE indicates a higher ability to prevent discoloration of hair.

### Dyed Wool Yarn Discoloration

A string of commercially available wool yarn (red) was cut into 10 cm pieces, which were bundled into a strand weighing 1 g. The wool strand was placed in a 100 ml bottle. Meanwhile, a test composition was diluted with distilled water to make a 0.5% aqueous solution. 100 ml of this solution was poured into the bottle. The bottle was then sealed with a cap and agitated for 2 days at room temperature.

Following the agitation period, the bottle was uncapped and the wool strand was pulled out. The wool strand was then washed thoroughly with water and was dried with a hair drier. The dried wool strand was analyzed by a differential calorimeter (Spectrophotometer SE 2000, NIPPON DENSHOKU Co. Ltd.) to determine the color difference (ΔE). A smaller ΔE indicates a higher ability to prevent discoloration of hair.

TABLE 5
Figures are concentrations.

| | | Comparative | |
|---|---|---|---|
| | Example | Example | |
| | 5 | 5 | 6 |
| Amphoteric surfactant of Production | 15 | | |
| Example 1 | | | |
| SLES | | 15 | 15 |
| pH conditioner | pH = 6 | pH = 6 | pH = 6 |
| Arginine | | 3 | |
| Purified water | to 100 | to 100 | to 100 |
| Stability at low temperature | ◯ | ◯ | ◯ |
| Ratings of hair manicure removal | 41.3 | 16.5 | 20.2 |
| performance | | | |
| Ratings of hair color removal performance | 2.5 | 16.7 | 22 |
| Ratings of dyed wool yarn decoloration | 0.9 | 6.3 | 6.5 |
| performance | | | |

The results of Table 5 demonstrate that the shampoo composition containing the amphoteric surfactant of the present invention can effectively remove hair manicure, or any foreign matter that adheres to the surface of hair, while it causes less discoloration of hair colors and dyed wool yarn. Thus, the shampoo composition causes less discoloration of dyed keratin products over a wide range of pH ranging from acidic to weakly basic. This is a unique performance not seen in conventional surfactants, making the detergent composition applicable in a wide range of pH.

### Example 6, and Comparative Examples 7 Through 12

We now describe the ability of the amphoteric surfactant of the general formula (1) to capture chlorine and chemical formulations that use the amphoteric surfactant.

The compound of Production Example 1, amidoacetic acidbetaines, each a surfactant having a similar structure to the compound of Production Example 1, and a conventional chlorine-capturing agent taurine were compared with each other for their ability to capture chlorine.

### Test Method

A sodium hypochlorite solution (First grade reagent, KANTO CHEMICAL Co. Ltd. Assay (as active chlorine min. 5.0%)) was added to 1 L of distilled water to adjust the concentration of free chlorine to 5mg/L (5 ppm). To this solution, 1.00 g of one of the surfactants was added (0.10 g of taurine was added in Comparative Example 7) and the concentrations of total residual chlorine and free chlorine were measured 30 min, 60 min and 90 min after addition of the surfactant.

The chlorine concentrations were determined by the diethyl-p-phenylenediamine (DPD) method, as follows.

Using a water testing kit (SIBATA SCIENTIFIC TECHNOLOGY Co. Ltd.), the surfactant solutions were colored corresponding to the chlorine concentration: The concentrations of residual chlorine and free chlorine at each time point were determined by comparing with a reference color chart.

TABLE 6
Cl conc. Unit: mg/L

| | Example 6 | Comparative Example | | | | | |
|---|---|---|---|---|---|---|---|
| | Compound of | | 8 | 9 | 10 | 11 | 12 |
| | Production | 7 | Softazoline | Softazoline | Softazoline | Soypon | Soypon |
| | Example 1 | Taurine | LAO Note 1 | LPB Note 2 | LSB Note 3 | SLE Note 4 | SLTA Note 5 |
| Free Cl conc. | 5 | 5 | 5 | 5 | 5 | 5 | 5 |
| Free Cl conc. immediately | 0.6 | 0.1 | 0.2 | 0.8 | 1 | 5 | 0.8 |
| after addition | | | | | | | |
| Free Cl conc. at 30 min | 0.2 | 0.1 | 0.1 | 0.4 | 1 | 5 | 0.4 |
| Free Cl conc. at 60 min | 0.1 | 0 | 0.1 | 0.4 | 0.4 | 5 | 0.4 |
| Free Cl conc. at 90 min | 0.1 | 0 | 0.1 | 0.2 | 0.2 | 5 | 0.2 |
| Total Cl conc. | 5 | 5 | 5 | 5 | 5 | 5 | 5 |
| Total Cl conc. immediately | 2 | 5 | 5 | 5 | 5 | 5 | 5 |
| after addition | | | | | | | |
| Total Cl conc. at 30 min | 2 | 5 | 5 | 5 | 5 | 5 | 5 |
| Total Cl conc. at 60 min | 2 | 5 | 5 | 5 | 5 | 5 | 5 |
| Total Cl conc. at 90 min | 2 | 5 | 5 | 5 | 5 | 5 | 5 |

Notes 1-5: Trade names of surfactants manufactured by Kawaken Fine Chemicals Co. Ltd.
Note 1: Softazoline LAO = lauroylamidopropylamine oxide
Note 2: Softazoline LPB = lauroylamidopropyl acetic acid betaine
Note 3: Softazoline LSB = lauroylamidopropyl hydroxyl sulfobetaine
Note 4: Soypon SLE = sodium lauroyl sarcosinate
Note 5: Soypon SLTA = triethanolamine lauroyl sarcosinate

As indicated by the results, the total chlorine level was decreased over time in the solution of the compound of Production Example 1, as was the free chlorine level. This is not because the total residual chlorine was decreased, but because chlorine captured by the compound of the general formula (1) was not released, resulting in an apparent decrease in the total residual chlorine level.

This observation indicates that once the compound of the general formula (1) captures chlorine, it will not readily release the chlorine. Thus, the compound of the general formula (1) can serve as an effective chlorine-capturing agent. Sodium lauroyl acylsarcosinate had no reactivity with free chlorine. Free chlorine was decreased in the solutions of the other surfactants and taurine with no significant changes in total chlorine concentration. This suggests that while these compounds can capture chlorine, they tend to release chlorine fairly easily.

### Example 7, and Comparative Examples 13 Through 17

### Effects on Hair Structure

A sodium hypochlorite solution (First grade reagent, KANTO CHEMICAL Co. Ltd. Assay (as active chlorine min. 5.0%)) was added to 1L of distilled water to make a solution with a free chlorine concentration of 5 mg/L (5 ppm). Each of the compounds shown in the table below was added to this solution or distilled water to a concentration of 100 mg/L (100 ppm). To these solutions, 10 g hair was immersed and was taken out. The hair was then rinsed three times with 1 L ion-exchanged distilled water and was then allowed to dry. The dried hair was observed by SEM.

For physical stress, samples were agitated on a table at 50 rpm.

SEM observation was evaluated based on the following criteria: A cross indicates that the cuticle layer was entirely peeled; a triangle indicates that some of the cuticle were peeled; and a circle indicates that no significant damage was observed.

TABLE 7
Unit mg/L

| | Comparative Example | | | | | Example |
|---|---|---|---|---|---|---|
| | 13 | 14 | 15 | 16 | 17 | 7 |
| Physical stress | Yes | No | Yes | Yes | Yes | Yes |
| Hypochlorous | No | Yes | Yes | Yes | Yes | Yes |
| acid | | | | | | |
| Taurine | | 100 | | 100 | | |
| Soypon SLE | | | | | 100 | |
| Compound of | | | | | | 100 |
| Production | | | | | | |
| Example 1 | | | | | | |
| Photo No | Photo | Photo 2 | Photo 3 | Photo 4 | Photo 5 | Photo 6 |
| | 1 | | | | | |
| SEM | ◯ | ◯ | X | Δ | X | ◯ |
| observation | | | | | | |

As can be seen from Table 7, a comparison between Comparative Examples 13, 14 and 16 indicates that although the addition of taurine, a chlorine-capturing compound, prevents the peeling of cuticle caused by free chlorine, application of physical stress rapidly decreases the advantageous effect of taurine (Comparative Example 16). In comparison, the chlorine-capturing agent of the present invention can protect the cuticle layer against physical stress (Example 7). No significant damage was observed in the hair treated with the chlorine-capturing agent of the present invention as compared to the treatment with distilled water (Comparative Example 13).

SEM photographs of the cuticles are shown in FIGURE.

Given below are exemplary formulations containing the amphoteric surfactant of the general formula (1) and other ingredients. These other ingredients are added in amounts that do not affect the viscoelastic characteristic or the foaming performance of the amphoteric surfactant, or the ability of the surfactant to prevent discoloration of dyed keratin products. The proportion of each ingredient is given as a percent by weight of solid component.

Each formulation is prepared as follows: All of the ingredients but perfume are weighed and mixed together. The mixture is heated to 80° C., is kept at the temperature for 10 min, and is subsequently allowed to cool to 60° C., at which point the perfume is added and the mixture is further cooled to room temperature to give the desired formulation.

### Example 8

### Shampoo for Dyed Hair

| | |
|---|---|
| Sodium polyoxyethylene (2) lauryl ether sulfate | 3.0 |
| (Note 6) | |
| Sodium polyoxyethylene (3) lauroylmonoethaol | 5.0 |
| amidosulfonate (30%) | |
| Coconut fatty acid diethanolamide | 1.5 |
| Amphoteric surfactant of Production Example 1 | 60.0 |
| (29.1%) | |
| Polyquartanium-7 | 0.5 |
| Carboxymethylsuccinyl chitosan solution | 1.0 |
| (Note 7) | |
| Citric acid | Amount to make |
| | pH of 6.5 |
| Glycerol | 1.5 |
| Perfume | As desired |
| Dipotassium glycyrrhizinate | 0.1 |
| Purified water | To 100% |

The proportion of the amphoteric surfactant of Production Example 1 was 82%. The concentration of the surfactant was 21.2%. The formulation scored 4.1 in the hair color discoloration test.
(Note 6)
Kao Co. Ltd. Emal 20C
(Note 7)
Kawaken Fine Chemicals Co. Ltd. Chitoaqua

Given below are exemplary formulations containing the amphoteric surfactant of the general formula (1) and other ingredients. These other ingredients are added in amounts that do not affect the viscoelastic characteristic or the foaming performance of the amphoteric surfactant, or the ability to capture chlorine. Different chemical formulations were prepared according to the formulas given below. As in Example 7, the shampoo compositions were tested for their effects on the cuticle and were proven to give no damage to cuticle.

The proportion of each ingredient is given as a percent by weight of solid component.

### Example 9

### Clear Anti-Chlorine Damage Shampoo

| | |
|---|---|
| Amphoteric surfactant of Production Example 1 | 10.00 |
| Triethanol lauryl sulfate | 7.00 |
| Viscosafe LPE (Note 8) | 2.00 |
| Sorbitan triisostearate PEG-160 | 0.80 |
| POE(50) hydrogenated castor seed oil monoisostearate | 1.00 |
| Glucosyltrehalose | 2.00 |
| Cationic cellulose | 0.20 |
| Octopirox | 0.75 |
| Salicylic acid | 0.20 |
| Taurine | 0.20 |
| Chitoaqua (Note 7) | 0.50 |
| Citric acid | 0.50 |
| Malic acid | 0.30 |
| Perfume | 0.50 |
| Purified water | To 100% |

(Note 8)
Lauryl glycol hydroxylpropyl ether Kawaken Fine Chemicals Co. Ltd.
(Note 7)
Succinyl carboxymethylchitosan solution Kawaken Fine Chemicals Co. Ltd.

### Example 10

### Anti-Chlorine Damage Shampoo Pearl

| | |
|---|---|
| Amphoteric surfactant of Production Example 1 | 10.00 |
| Sodium POE(2) lauryl ether sulfate | 6.50 |
| Sodium laurate | 1.50 |
| Amizett 1PC (Note 9) | 2.50 |
| Glycerol | 2.00 |
| Ethylene glycol distearate | 1.00 |
| Taurine | 0.10 |
| Glycolic acid | 0.20 |
| Japanese green gentian extract | 0.01 |
| Carrot extract | 0.01 |
| Perfume | 0.30 |
| Purified water | To 100% |

(Note 9)
POE (1) coconut oil fatty acid monoisopropanol amide Kawaken Fine Chemicals Co. Ltd.

### Example 11

### Clear Anti-Chlorine Damage Shampoo

| | |
|---|---|
| Amphoteric surfactant of Production Example 1 | 12.00 |
| Soypon SCTA (Note 10) | 4.00 |
| Mydol 10 (Note 11) | 8.00 |
| Viscosafe LMPE (Note 12) | 1.70 |
| Sorbitan triisostearate PEG-160 | 0.50 |
| Glucosyltrehalose | 2.00 |
| Cationic cellulose | 0.20 |
| Cationic guar | 0.20 |
| Taurine | 0.20 |
| Salicylic acid | 0.20 |
| Octopirox | 0.75 |
| Citric acid | 0.50 |
| Perfume | 0.50 |
| Purified water | To 100% |

(Note 10)
Coconut oil fatty acid sarcosine triethanolamine Kawaken Fine Chemicals Co. Ltd.
(Note 11)
Surfactant composed mainly of alkyl glucoside KAO Co. Ltd.
(Note 12)
(Lauryl/myristyl)glycol hydroxypropylether Kawaken Fine Chemicals Co. Ltd.

### Example 12

### Anti-Chlorine Damage Solid Detergent

| | |
|---|---|
| Polyquartanium-10 | 0.35 |
| Cocoylglutamic acid | 0.50 |
| Sodium POE(2) lauryl ether sulfate | 7.00 |
| Amphoteric surfactant of Production Example 1 | 6.00 |
| Lauryl dimethyl acetic acid betaine | 3.00 |
| Amizett 1PC (Note 9) | 3.00 |
| Polyquartanium-7 | 0.75 |
| Tornare (Note 13) | 1.00 |
| Amiter LGOD-5(H) (Note 14) | 0.50 |
| Ethylene glycol distearate | 2.00 |
| Methyl paraben | 0.20 |
| Propyl paraben | 0.10 |
| EDTA-2Na | 0.20 |
| Perfume | 0.30 |
| Purified water | To 100% |

(Note 13)
Cosmetic base containing glycosyltrehalose HAYASHIBARA BIOCHEMICAL LAB Co. Ltd.
(Note 14)
Dioctyldodeceth-5 lauroyl glutamate AJINOMOTO Co., Inc.

### Example 13

### Anti-Chlorine Damage Body Soap

| | |
|---|---|
| Lauric acid | 3.00 |
| Myristic acid | 6.80 |
| Palmitic acid | 1.30 |
| Potassium hydroxide | 2.95 |
| Amphoteric surfactant of Production Example 1 | 5.60 |
| Kawasilk S (Note 15) | 4.00 |
| Viscosafe LMPE (Note 12) | 2.00 |
| Glycerol | 5.00 |
| Pyroter CPI-40 (Note 16) | 0.50 |
| Ethylene glycol distearate | 2.00 |
| EDTA-2Na | 0.20 |
| Methyl paraben | 0.20 |
| Propyl paraben | 0.10 |
| Perfume | 0.20 |
| Purified water | To 100% |

(Note 15)
Lauroyl hydrolyzed silk amino acid potassium salt Kawaken Fine Chemicals Co. Ltd.
(Note 16)
PCA isostearic acid PEG-40 hydrogenated castor seed oil AJINOMOTO Co., Inc.

### Example 14

### Anti-Chlorine Damage Face Wash Gel

| | |
|---|---|
| Potassium salt of coconut fatty acid | 10.00 |
| Amphoteric surfactant of Production Example 1 | 15.00 |
| Lauryl dimethyl acetic acid betaine | 2.00 |
| Viscosafe LPE (Note 8) | 0.15 |
| Glycerol | 1.00 |
| Raffinose | 1.00 |
| Trimethylglycine | 0.50 |
| EDTA-4Na | 0.20 |
| Methyl paraben | 0.20 |
| Propyl paraben | 0.10 |
| Perfume | 0.10 |
| Potassium hydroxide | 0.25 |
| Purified water | To 100% |

### Example 15

### Anti-Chlorine Damage Solid Detergent

| | |
|---|---|
| Amisoft GS-11 (Note 17) | 78.90 |
| Cetanol | 5.00 |
| Behenyl alcohol | 2.00 |
| Isostearyl alcohol | 1.00 |
| Compound of Production Example 1 | 3.00 |
| Titanium oxide | 0.10 |
| Purified water | 10.00 |

(Note 17)
Mixed fatty acid acylglutamic acid Na salt (Powder) AJINOMOTO Co., Inc.

Surfactants of the present invention represented by the general formula (1) reduce adverse effects of free chlorine and ensure high color retention of dyed hair, dyed keratin fibers and dyed fabrics made of dyed keratin fibers. Thus, the surfactants of the present invention are suitable for use in various chemical formulations, such as detergents to prevent discoloration of dyed hair and dyed keratin fibers, cosmetics and post-treatment agents for bleached products.

1-8. (canceled)

9. A method for capture of chlorine, comprising,
contacting chlorine dissolved solution or the materials including chlorine with a chlorine-capturing agent comprising the amphoteric surfactant represented by the following general formula (1) 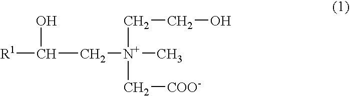
wherein R¹ is an alkyl or alkenyl group having 10 to 18 carbon atoms, or mixing the chlorine dissolved solution or the materials including chlorine with the chlorine-capturing agent.

10. The method according to claim 9, wherein the chlorine-capturing agent captures free chlorine in shampooing human hair.

11. The method according to claim 10, wherein the human hair is dyed human hair.

12. The method according to claim 9, wherein the chlorine-capturing agent captures free chlorine in washing dyed wool.
